(19) United States
(12) Patent Application Publication
(10) Pub. No.: US 2026/0117445 A1
(43) Pub. Date: Apr. 30, 2026

(54) CONTROL ASSEMBLY AND METHOD FOR AN APPLIANCE USING A NEAR INFRARED SPECTROSCOPE

(71) Applicant: TE Connectivity Solutions GmbH, Schaffhausen (CH)

(72) Inventor: Robin Wesson, Middletown, PA (US)

(21) Appl. No.: 18/926,861

(22) Filed: Oct. 25, 2024

(51) Int. Cl. D06F 34/22; D06F 23/02; D06F 33/38; D06F 34/08; D06F 34/30; D06F 103/22; D06F 105/02; D06F 105/08; D06F 105/46; D06F 105/52; G01N 21/3577; G01N 21/359; G01N 33/18

(52) U.S. Cl. CPC D06F 34/22; D06F 23/02; D06F 33/38; D06F 34/08; D06F 34/30; G01N 21/3577; G01N 21/359; G01N 33/18; D06F 2103/22; D06F 2105/02; D06F 2105/08; D06F 2105/46; D06F 2105/52

(57) ABSTRACT

A control assembly for an appliance includes a control panel for the appliance. The control panel includes inputs for controlling the appliance. The control assembly includes a control circuit board coupled to the control panel. The circuit board configured to be operably coupled to working components of the appliance to control operation of the appliance based on the inputs from the control panel. The control assembly includes a detergent sensor configured to sense wash characteristics of water in the appliance. The detergent sensor includes a near infrared spectroscope (NIRS) for determining optical properties of a sample of the water. The detergent sensor performs a chemical analysis to determine the contents of the sample based on the optical properties. The control circuit board is configured to control the working components of the appliance based on the chemical analysis performed by the detergent sensor.

### BACKGROUND OF THE INVENTION

The subject matter herein relates generally to a control assembly and method for an appliance.

Washing machines have many programs each with different combinations of temperature, agitation, spin speed, rinse cycles and time to complete amongst other parameters. In practice, users need to correctly map the array of options to each load, understanding the material types, volume and soilage condition. Their ability to do this accurately affects their experience of, and satisfaction with, the appliance. It is in the manufacturers interest to make the matching of program to load as easy as possible, preferably automated and hence foolproof.

One specific aspect involved in the program definition is how many rinse cycles are performed. Detergent washes out of clothing at different rates depending on many factors including the fiber types, clothing volume, and detergent type and volume applied. Also the chemical process of washing (grease and detergent molecules binding and being attracted by water) impacts the detergent wash out rate. Given these variables in real world use, it is complex for manufacturers to design programs that balance the number of rinse cycles with the environmental impact of water usage. Water use, along with electrical efficiency are increasingly becoming a consideration at point of sale.

There is a need for sensors that can automatically detect when the water rinsed out of a machine is sufficiently clear of detergent.

### BRIEF DESCRIPTION OF THE INVENTION

In one embodiment, a control assembly for an appliance is provided and includes a control panel for the appliance. The control panel includes inputs for controlling the appliance. The control assembly includes a control circuit board coupled to the control panel. The circuit board configured to be operably coupled to working components of the appliance to control operation of the appliance based on the inputs from the control panel. The control assembly includes a detergent sensor configured to sense wash characteristics of water in the appliance. The detergent sensor includes a near infrared spectroscope (NIRS) for determining optical properties of a sample of the water. The detergent sensor performs a chemical analysis to determine the contents of the sample based on the optical properties. The control circuit board is configured to control the working components of the appliance based on the chemical analysis performed by the detergent sensor.

In another embodiment, an appliance is provided and includes a housing. The appliance includes a drum rotatable in the housing, a motor operably coupled to the drum to rotate the drum, a water valve for filling the drum through a water inlet and a water pump for draining the drum through a drain pipe. The appliance includes a control assembly for controlling the motor, the water valve, and the water pump. The control assembly includes a control panel mounted to the housing. The control panel includes inputs for controlling the appliance. The control assembly includes a control circuit board coupled to the control panel. The control circuit board configured to be operably coupled to the motor, the water valve, and the water pump to control operation of the appliance based on the inputs from the control panel. The control assembly includes a detergent sensor configured to sense wash characteristics of water in the appliance. The detergent sensor includes a near infrared spectroscope (NIRS) for determining optical properties of a sample of the water. The detergent sensor performs a chemical analysis to determine the contents of the sample based on the optical properties. The control circuit board is configured to control the working components of the appliance based on the chemical analysis performed by the detergent sensor.

In a further embodiment, a method of controlling an appliance is provided. The method provides a detergent sensor in fluid communication with an internal chamber of the appliance to sense wash characteristics of water in the appliance. The detergent sensor includes a near infrared spectroscope (NIRS) for determining optical properties of a sample of the water. The method performs a chemical analysis of the sample based on the optical properties to determine the contents of the sample. The method sends a sensor signal from the detergent sensor to a control circuit board relating to the chemical analysis and sends a control signal from the control circuit board to at least one working component of the appliance to control operation of the appliance based on the chemical analysis performed by the detergent sensor.

In a further embodiment, the detergent sensor includes a minimum number of wavelengths necessary to discriminate the levels of detergent needed for an application. Such a ‘Minimal Viable Product’ sensor may have for example 3-30 wavelengths rather than the hundreds or thousands needed for laboratory grade NIRS measurements. The minimum number of wavelengths being implementable with low cost light emitting diodes and a broadband optical detector to create a solution compatible with high volume appliance business cases. The minimum number of wavelengths may be based on laboratory testing of detergent samples and may be based on machine optimization techniques, allowing the detection mechanisms to be fine tuned to particular manufacturers priorities, programs and environmental and performance goals.

In various embodiments, the LED emitters can be placed in a circular arrangement around the central wideband photodetector/photodiode so that all emitters are spaced equally from the detector, allowing the optical characteristics of the turbid liquid containing detergent to be analyzed in reflection. In various embodiments, a detector can be opposed to the emitters so that transmission mode can be analyzed. In various embodiments, a detector can be placed off axis to detect scattered light. Multiple modalities can be sensed in one assembly to enable more robust analysis.

Data from the sensor forms a digital fingerprint which can be analyzed by a trained neural net which allows for the translation of the multivariate data into a vale indicating the presence, and quantity, of a type and quantity of detergent.

### BRIEF DESCRIPTION OF THE DRAWINGS

FIG. 1 is a schematic view of an appliance including a detergent sensor in accordance with an exemplary embodiment.

FIG. 2 is a schematic view of the appliance in accordance with an exemplary embodiment.

FIG. 3 is a schematic view of the detergent sensor in accordance with an exemplary embodiment.

FIG. 4 is a flowchart showing a method of controlling an appliance in accordance with an exemplary embodiment.

FIG. 5 is a schematic view of the detergent sensor in accordance with an exemplary embodiment.

FIG. 6 is a schematic view of the detergent sensor in accordance with an exemplary embodiment.

FIG. 7 is a schematic view of the detergent sensor in accordance with an exemplary embodiment.

FIG. 8 is a schematic view of the detergent sensor in accordance with an exemplary embodiment.

FIG. 9 is a schematic view of the detergent sensor in accordance with an exemplary embodiment showing example optical paths illuminated by a first sensor.

FIG. 10 is a schematic view of the detergent sensor in accordance with an exemplary embodiment showing example optical paths illuminated by a second sensor.

FIG. 11 is a schematic view of the detergent sensor in accordance with an exemplary embodiment showing example optical paths illuminated by a third sensor.

FIG. 12 is a schematic view of an artificial neural network for the detergent sensor in accordance with an exemplary embodiment.

FIG. 13 is a schematic view showing exemplary inputs to nodes of the artificial neural network for the detergent sensor in accordance with an exemplary embodiment.

### DETAILED DESCRIPTION OF THE INVENTION

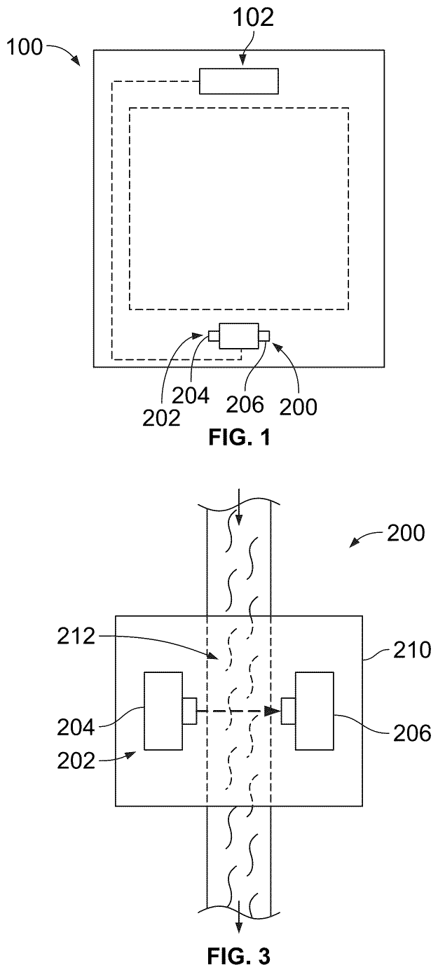

FIG. 1 is a schematic view of an appliance 100 including a detergent sensor 200 in accordance with an exemplary embodiment. In an exemplary embodiment, the detergent sensor 200 is configured to sense wash characteristics of water used in a wash cycle performed by the appliance 100. In various embodiments, the appliance 100 is a clothes washing machine. However, the detergent sensor 200 may be used in other types of appliances in alternative embodiments.

The appliance 100 includes a control assembly 102 for controlling the appliance 100. The detergent sensor 200 is an operable component of the control assembly 102. For example, control of the appliance 100 may be based on the wash characteristics sensed by the detergent sensor 200. Control of the appliance 100 may be based on analysis or processing of the wash characteristics sensed by the detergent sensor 200. In an exemplary embodiment, the wash cycle of the appliance 100 is controlled based on signals from the detergent sensor 200. For example, the length of a rinse cycle of the appliance 100 may be controlled based on signals from the detergent sensor 200. The number of rinse cycles of the appliance 100 may be controlled based on signals from the detergent sensor 200. In various embodiments, the control assembly 102 is configured to cease the rinse cycle of the appliance 100 based on the wash characteristics sensed by the detergent sensor 200 allowing the appliance 100 to run more efficiently and autonomously. For example, the rinse cycle may be ended early and/or the total number of rinse cycles for the appliance 100 may be reduced based on signals from the detergent sensor 200 thus reducing the total amount of water and electricity used in a wash, saving money for the consumer and improving environmental sustainability.

In an exemplary embodiment, the detergent sensor 200 includes a near infrared spectroscope (NIRS) 202. The NIRS 202 determines optical properties of the sample of the water used in the appliance 100. The detergent sensor 200 performs a chemical analysis to determine the contents of the sample based on the optical properties processed by the NIRS 202. The control assembly 102 is configured to control one or more of the working components of the appliance 100 based on the chemical analysis performed by the detergent sensor 200. For example, the control assembly 102 may control a water valve, a motor, or other working component of the appliance 100, such as to control a fill operation, a drain operation, an agitation operation, a spin operation, and the like of the appliance 100.

In an exemplary embodiment, the NIRS 202 includes a light source 204 and a photodetector 206 receiving light from the light source 204. The light is directed toward a sample of the water in the appliance. The light may be reflected by the sample and/or transmitted through the sample. Optionally, multiple photodetectors 206 may be provided, such as one or more receiving light reflected by the sample and one or more receiving light transmitted through the sample. The NIRS 202 analyzes one or more wavelengths of the light to perform a chemical analysis. In an exemplary embodiment, the NIRS 202 includes an artificial neural network that analyzes the wavelengths of light to perform the chemical analysis. The chemical analysis determines the contents (for example, information about what is in) the sample of the water. In an exemplary embodiment, the chemical analysis determines an amount of detergent in the water, which may correspond to the effectiveness and/or sufficiency of the rinse cycle. The chemical analysis may additionally or alternatively determine the type of detergent in the water. The chemical analysis may additionally or alternatively determine an amount of soil level in the water. The chemical analysis may additionally or alternatively determine an agitation level of the water. In an exemplary embodiment, the NIRS 202 may determine at least one of a transmittance, a reflectance, and an absorbance of the sample of the water. The NIRS 202 is used during the wash cycle to control the operation of the appliance in real time. The NIRS 202 is used to enhance the appliance 100 performance and energy and water savings. The chemical analysis performed by the NIRS 202 provide useful information about the contents in the water, such as the detergent and/or the soil in the water.

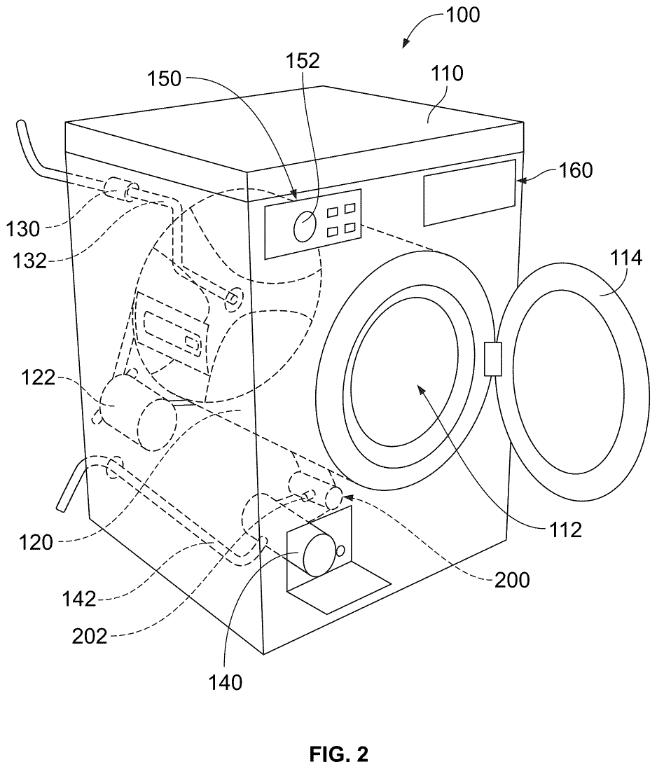

FIG. 2 is a schematic view of the appliance 100 in accordance with an exemplary embodiment. In the illustrated embodiment, the appliance 100 is a front-load clothes washing machine. The appliance 100 may be a top-load clothes washing machine in alternative embodiments. Other types of appliances may be provided in alternative embodiments.

The appliance 100 includes a housing 110 having an inner chamber 112 and a lid or door 114 configured to close the internal chamber 112. The appliance 100 includes a tub or drum 120 received in the inner chamber 112 of the housing 110. The drum 120 is rotatable within the housing 110. Clothes or laundry is configured to be loaded into the drum 120 in the door 114 is open. The appliance 100 includes an actuator 122 for rotating the drum 120. The actuator 122 may be an electric motor operably coupled to the drum 120, such as by a pulley. The appliance 100 includes a water valve 130 for filling the drum 120 through a water inlet 132. The appliance 100 includes a water pump 140 for draining the drum 120 through a drain pipe 142.

During use, laundry and detergent are added to the drum 120. The size of the load, how soiled the laundry is, and the type of detergent used can all influence the amount detergent needed and the number of wash/spin/rinse cycles that are required for optimal results. Typically, the detergent volume is defined independently of the state of the laundry and the number of spin and rent cycles are defined by a program set by the manufacturer. The washing machine manufacture can define a number of programs, but it is up to the user to select the optimal program, which is often based on guesswork or habit. The programs may be overdesigned to ensure an adequate number of wash/spin/rinse cycles to completely flush the detergent from the laundry, leading to increase water and electricity use in each wash. In an exemplary embodiment, the detergent sensor 200 monitors the contents of the water during each cycle and provides feedback to the control assembly 102. For example, the detergent sensor 200 is used to monitor the detergent level in the wash in real time to determine when the detergent level is at a level sufficient to end the wash cycle, thus saving water and electricity use by eliminating unnecessary wash/spin/rinse cycles. Reducing rinse cycles can significantly improve environmental sustainability without compromising performance of the appliance 100. The detergent sensor 200 is configured to detect performance of the appliance 100 and end the rinse cycle early to enhance the energy efficiency of the washing machine. The detergent sensor 200 may detect turbidity in the water to measure how clear the water is, which is a key indicator of the amount of detergent, contaminants, soil, and the like in the water. In an exemplary embodiment, the detergent sensor 200 analyzes wavelengths of light to perform a chemical analysis to determine the turbidity of the water, such as to determine a level or type of suspended particles in the water.

The appliance 100 includes the control assembly 102 including the detergent sensor 200. The control assembly 102 may include other types of sensors in addition to the detergent sensor 200, such as temperature sensors, water level sensors pressure sensors, vibration sensors, rotor position sensors, and the like. One or more of the sensors may be incorporated into a multi-sensor assembly.

The control assembly 102 includes a control panel 150 mounted to the housing 110 and a control circuit board 160 operably coupled to the control panel 150 in the detergent sensor 200. The control circuit board 160 is operably coupled (for example, wired or wireless connection) to one or more of the working components of the appliance 100, such as the actuator 122 and/or the water valve 130 and/or the water pump 140, to control one or more operating processes of the appliance 100. The control panel 150 includes inputs 152 for controlling the appliance 100. The inputs 152 may include buttons, dials, sliders, keypads, or other types of inputs. The inputs 152 receive commands, instructions, requests, or other types of inputs from the operator. The operator of the appliance 100 may adjust the inputs 152 to control the appliance 100. For example, the inputs 152 may relate to start/stop, load size, water temperature, rinse cycle, spin speed, and the like. The control panel 150 may include instruments, gauges, a display screen (e.g., screen, monitor, touch screen, heads up display (HUD), indicator light, etc.), or other type of output device to display readings or other parameters to the operator of the appliance 100.

The control circuit board 160 includes a control circuit or driver to facilitate operating the operable or working components of the appliance 100. For example, the controller control circuit board 160 includes a processing circuit having a processor and a memory. The processor may include a microprocessor, an application-specific integrated circuit (ASIC), a field-programmable gate array (FPGA), etc., or combinations thereof. The memory may include, but is not limited to, electronic, optical, magnetic, or any other storage or transmission device capable of providing a processor, ASIC, FPGA, etc. with program instructions. The memory may include a memory chip, Electrically Erasable Programmable Read-Only Memory (EEPROM), Erasable Programmable Read Only Memory (EPROM), flash memory, or any other suitable memory from which the control circuit can read instructions. The instructions may include code from any suitable programming language. The memory may include various modules that include instructions that are configured to be implemented by the processor.

The detergent sensor 200 may be positioned at one or more of multiple locations within the appliance 100. For example, the detergent sensor 200 may be located within the drum 120. The detergent sensor 200 may be located in the drain pipe 142. The detergent sensor 200 may be located within the door 114 (for example, in front load washing machines wherein the water is in contact with the door 114). In an exemplary embodiment, the detergent sensor 200 is located in a light tight section of the appliance 100 that water in the drum is able to flow through to perform optical testing of the sample solution, such as to monitor detergent concentration under operating conditions. For example, as the concentration of the detergent decreases, the intensity of the spectrum of the light sensed by the NIRS 202 also decreases, such as due to a decrease in opacity of the sample caused by diminished detergent level and/or diminished bubble production due to agitation of the water in the appliance. The NIRS 202 of the detergent sensor 200 is configured to shine light onto or through a sample of the water and measure the reflected and/or transmitted intensity of the light over a range of infrared wavelengths. The NIRS 202 takes spectral measurements of the sample solution and performs a chemical analysis of the sample solution. For example, the NIRS 202 may analyze the residual detergent content in the rinse water to allow the washing machine to run the required number of rinse cycles needed, and no more, resulting in improved performance, such as from water savings, energy savings, time savings, and the like. The NIRS 202 allows the washing machine to run more efficiently and autonomously for improved performance and enhanced consumer satisfaction.

In an exemplary embodiment, the detergent sensor 200 includes a minimum number of wavelengths necessary to discriminate the levels of detergent needed for an application. The detergent sensor 200 is a minimal viable product sensor having, for example, between 3-30 wavelengths, as compared to the hundreds or thousands of wavelengths of laboratory grade NIRS measurements. The minimum number of wavelengths being implementable with low cost light emitting diodes and a broadband optical detector creates a solution compatible with high volume appliance business cases. The minimum number of wavelengths may be based on laboratory testing of detergent samples and may be based on machine optimization techniques, allowing the detection mechanisms to be fine-tuned to particular manufacturers priorities, programs and environmental and performance goals.

In various embodiments, the LED emitters can be placed in a circular arrangement around a central wideband photodetector/photodiode so that all emitters are spaced equally from the detector, allowing the optical characteristics of the turbid liquid containing detergent to be analyzed in reflection. Other arrangements of LED emitters and photodetectors may be used in alternative embodiments. In various embodiments, a detector can be opposed to the emitters so that a transmission mode (as opposed to a refection mode) can be analyzed. In various embodiments, a detector can be placed off axis to detect scattered light. Multiple modalities can be sensed in one assembly to enable more robust analysis.

Data from the detergent sensor 200 forms a digital fingerprint which can be analyzed by a trained neural net which allows for the translation of the multivariate data into a vale indicating the presence, and quantity, of a type and quantity of detergent.

FIG. 3 is a schematic view of the detergent sensor 200 in accordance with an exemplary embodiment. The detergent sensor 200 includes a sensor housing 210 having a fluid channel 212 configured to receive a sample of the water therein. In an exemplary embodiment, the fluid channel 212 passes through the sensor housing 210 to allow fluid flow through the sensor housing 210. The detergent sensor 200 may be incorporated into a multi-sensor assembly having multiple sensors incorporated into the single sensor housing 210. For example, temperature sensors, pressure sensors, water level sensors, or other types of sensors may be incorporated into the sensor assembly.

The detergent sensor 200 includes the NIRS 202. The NIRS 202 includes the light source 204 and the photodetector 206. The light source 204 transmits light at various wavelengths and output emission ranges, such as infrared, near infrared, visible light, ultraviolet light, or other wavelength ranges. The light source 204 may transmit light at a range of between 360 nm and 2700 nm. In various embodiments, the light source 204 may be a broadband tungsten halogen bulb. However, other types of light sources may be used in alternative embodiments. The photodetector 206 may be a photodiode or a phototransistor. In an exemplary embodiment, the photodetector 206 may be a near infrared spectroscopy photodiode, such as a germanium photodiode, a lead sulfide photodiode, a silicone photodiode, and indium arsenide photodiode, or another type of photodiode.

In the illustrated embodiment, the light source 204 and the photodetector 206 are arranged on opposite sides of the fluid channel 212 such that the sample of the water is located between the light source 204 and the photodetector 206. The photodetector 206 is configured to receive the light transmitted through the sample from the light source 204 to the photodetector 206. For example, such arrangement is operable in a transmission mode configured to receive light from the light source 204 transmitted through the sample to measure a transmittance and/or absorbance of the sample. In other various embodiments, the photodetector 206 may be located on the same side of the fluid channel 212 as the light source 204 configured to receive light that is reflected by the sample from the light source 204. For example, such arrangement is operable in a reflection mode configured to receive light from the light source 204 reflected by the sample to measure a reflectance and/or absorbance of the sample. In various embodiments, the NIRS 202 includes multiple photodetectors 206, such as having one or more on the same side of the fluid channel 212 as the light source 204 and one or more on the opposite side of the fluid channel 212 from the light source 204.

The NIRS 202 includes one or more processors determining optical properties of the sample of the water in the fluid channel 212. For example, the NIRS 202 includes one or more processors analyzing wavelengths of the light received at the photodetector 206. In various embodiments, the NIRS 202 includes one or more processors analyzing intensity of the light received at the photodetector 206. In various embodiments, the NIRS 202 includes one or more processors analyzing transmission of the light received at the photodetector 206. In various embodiments, the NIRS 202 includes one or more processors analyzing absorbance of the light received at the photodetector 206. The optical properties of the sample of the water are affected by various factors such as the volume of the water in the appliance, the volume and/or type of materials in the laundry, the agitation level of the wash cycle, the amount of soil or contaminants suspended in the water, the amount or value of the detergent, the type of detergent, and the like. The chemical analysis performed by the NIRS 202 is used to determine one or more sensor outputs relating to the water level, the detergent level and/or the soil level, which is used to control operation of the appliance 100.

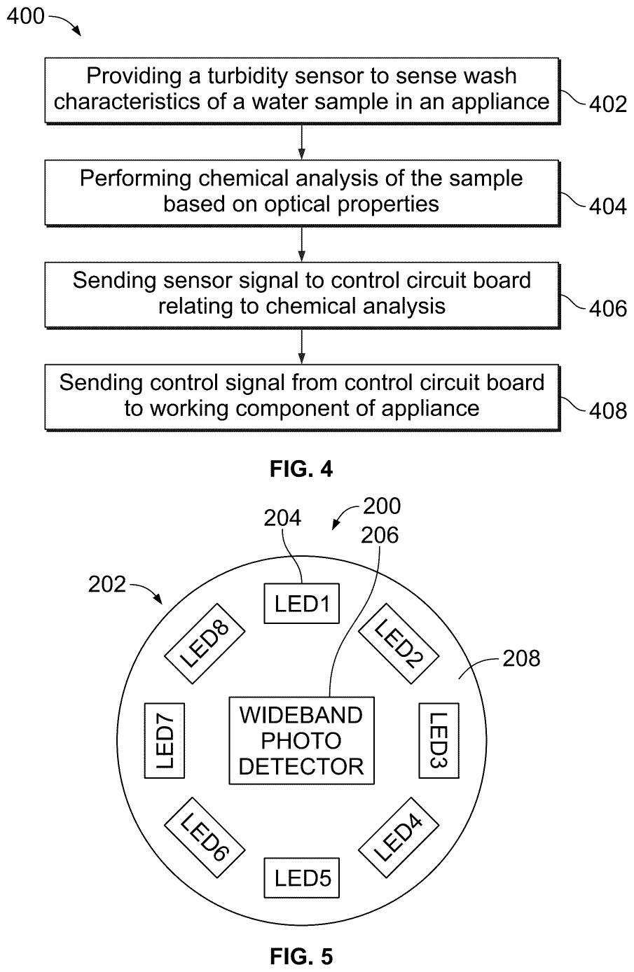

FIG. 4 is a flowchart showing a method 400 of controlling an appliance in accordance with an exemplary embodiment.

At 402, the method includes providing a detergent sensor in fluid communication with an internal chamber of the appliance to sense wash characteristics of water in the appliance. The detergent sensor includes a NIRS for determining optical properties of a sample of the water. For example, the detergent sensor includes a photodetector receiving light from a light source to detect optical properties of the light interacting with the sample (for example, reflecting off of the sample or transmitting through the sample), such as the intensity, frequency, transmittance, reflectance, absorbance, or other optical properties. The photodetector may be operable in a reflection mode receiving light from the light source reflected by the sample. The photodetector may be operable in a transmission mode receiving light from the light source transmitted through the sample.

At 404, the method includes performing a chemical analysis of the sample based on the optical properties. The chemical analysis is performed to determine the contents of the sample. In an exemplary embodiment, the chemical analysis is performed to determine or measure a detergent content in the sample. The chemical analysis may be performed to determine an amount of detergent in the water. The chemical analysis may be performed to determine a type of detergent in the water, such as a brand of detergent or if the detergent is granular or liquid. The chemical analysis may be performed to determine at least one of a transmittance, a reflectance, and an absorbance of the sample. The chemical analysis may be performed using an artificial neural network to analyze the optical properties.

At 406, the method includes sending a sensor signal from the detergent sensor to a control circuit board relating to the chemical analysis. The sensor signal may be a raw signal or a processed signal. The sensor signal may be transmitted by a wired connection or a wireless connection.

At 408, the method includes sending a control signal from the control circuit board to at least one working component of the appliance to control operation of the appliance based on the chemical analysis performed by the detergent sensor. In various embodiments, the control signal may be used to control the motor that rotates the drum of the washing machine to start or stop the motor or change the speed and/or direction of the motor. The control signal may be used to control the water pump to initiate a drain cycle. The control signal may be used to control the water inlet to initiate a water fill, such as for a rinse cycle. The control signal may be sent to the control system of the appliance to perform an additional rinse cycle based on the chemical analysis, such as relating to the detergent level in the sample.

FIG. 5 is a schematic view of the detergent sensor 200 in accordance with an exemplary embodiment. The NIRS 202 of the detergent sensor 200 is a multi-sensor assembly. The detergent sensor 200 includes a substrate 208 that supports the light source(s) 204 and the photodetector(s) 206.

In an exemplary embodiment, the detergent sensor 200 includes an array of light sources 204 (for example, emitters), such as LEDs, arranged in a pattern on a surface of the substrate 208. For example, in the illustrated embodiment, the detergent sensor 200 includes eight of the LEDs 204 arranged circumferentially around a wideband photodetector 206. The number of light sources 204 can be tuned to the particular application. In an exemplary embodiment, the detergent sensor 200 includes greater than two light sources 204. The number of light sources 204 may be limited to control costs of the detergent sensor 200 (for example, fewer light sources than used in laboratory NIRS systems). The light sources 204 transmit light at various wavelengths and output emission ranges, such as infrared, near infrared, visible light, ultraviolet light, or other wavelength ranges. The light sources 204 may transmit light at a range of between 360 nm and 2700 nm.

In an exemplary embodiment, the substrate 208 is circular. However, the substrate 208 may have other shapes in alternative embodiments. In an exemplary embodiment, the photodetector 206 is centered on the circular substrate 208 and the LEDs 204 may be arranged at a perimeter of the substrate 208. The LEDs 204 may be arranged equidistant from the photodetector 206. Other shapes and arrangements may be used in alternative embodiments. The photodetector 206 may be a photodiode or a phototransistor. In an exemplary embodiment, the photodetector 206 may be a near infrared spectroscopy photodiode, such as a germanium photodiode, a lead sulfide photodiode, a silicone photodiode, and indium arsenide photodiode, or another type of photodiode.

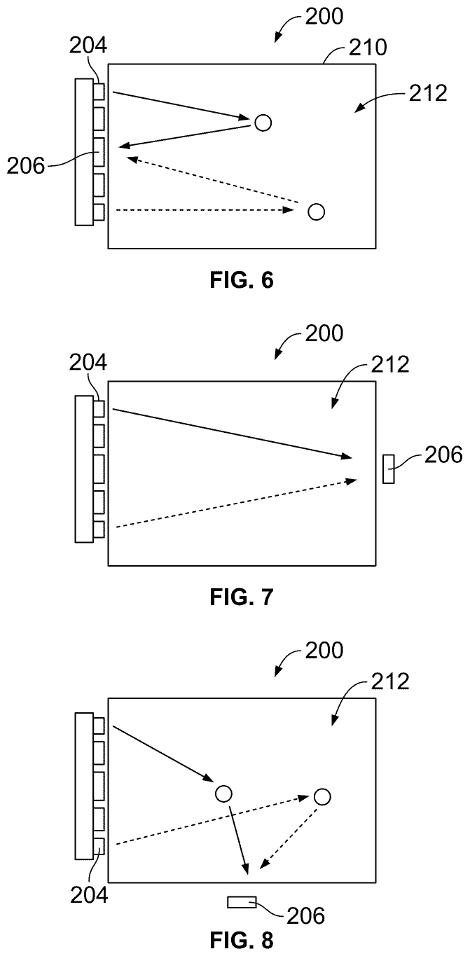

FIG. 6 is a schematic view of the detergent sensor 200 in accordance with an exemplary embodiment. The detergent sensor 200 includes the sensor housing 210 having the fluid channel 212 configured to receive a sample of the water therein. In an exemplary embodiment, the fluid channel 212 passes through the sensor housing 210 to allow fluid flow through the sensor housing 210. The detergent sensor 200 includes the light sources 204 and the photodetector 206 arranged on the substrate 208.

In the illustrated embodiment, the light sources 204 and the photodetector 206 are arranged on a side of the fluid channel 212. The photodetector 206 is located on the same side of the fluid channel 212 as the light sources 204. The photodetector 206 is configured to receive light that is reflected by the sample from the light source 204. For example, light that is refracted or reflected from the liquid and the suspended content is transmitted to and detected by the photodetector. Such an arrangement is operable in a reflection mode configured to receive light from the light sources 204 reflected by the sample to measure a reflectance and/or absorbance of the sample.

FIG. 7 is a schematic view of the detergent sensor 200 in accordance with an exemplary embodiment. In the illustrated embodiment, the detergent sensor 200 includes the photodetector 206 at the opposite side of the fluid channel 212 from the light sources 204. The photodetector 206 is configured to receive light that is transmitted through the sample from the light sources 204. For example, such arrangement is operable in a transmission mode configured to receive light from the light source 204 transmitted through the sample to measure a transmittance and/or absorbance of the sample.

FIG. 8 is a schematic view of the detergent sensor 200 in accordance with an exemplary embodiment. In the illustrated embodiment, the detergent sensor 200 includes the photodetector 206 at a different side of the fluid channel 212 from the light sources 204, such as the bottom of the fluid channel 212. The photodetector 206 is configured to receive light that is refracted or reflected from the liquid and the suspended content is transmitted to and detected by the photodetector.

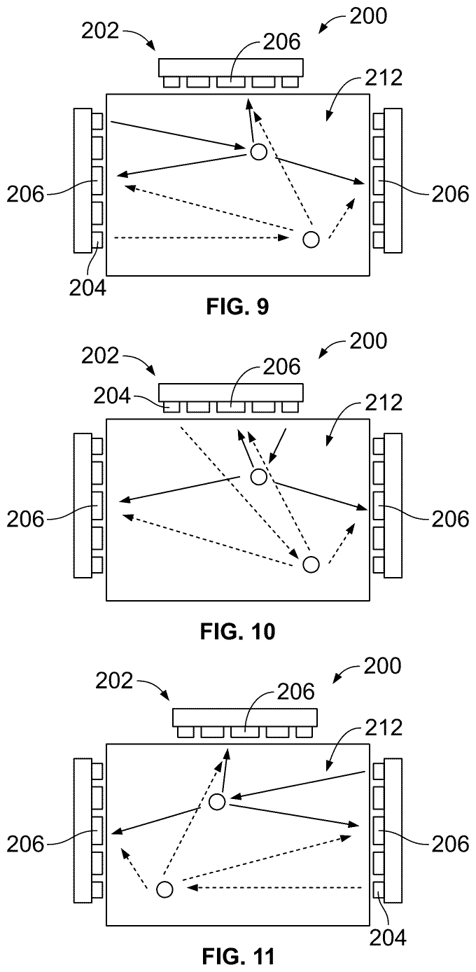

FIG. 9 is a schematic view of the detergent sensor 200 in accordance with an exemplary embodiment showing example optical paths illuminated by a first sensor. FIG. 10 is a schematic view of the detergent sensor 200 in accordance with an exemplary embodiment showing example optical paths illuminated by a second sensor. FIG. 11 is a schematic view of the detergent sensor 200 in accordance with an exemplary embodiment showing example optical paths illuminated by a third sensor. The detergent sensor 200 includes multiple NIRS assemblies 202 arranged at different sides of the fluid channel 212. Each NIRS 202 includes the corresponding light sources 204 and the photodetector 206 arranged on the corresponding substrate 208.

Providing multiple optical paths allows transmission of light in different directions and sensing of light from different directions to provide the detergent sensor 200 with a sense of agitation level and suspended contaminants in the sample. The multiple sensors, arranged at different sides of the fluid channel 212, provide reliable turbidity sensing for the sample allowing transmission sensing, reflection sensing, and refraction sensing. The data from the sensors may be analyzed in an artificial neural network, such as to determine an amount of bubble formation, cavitation, soil particles or other chemicals detectable by the detergent sensor 200.

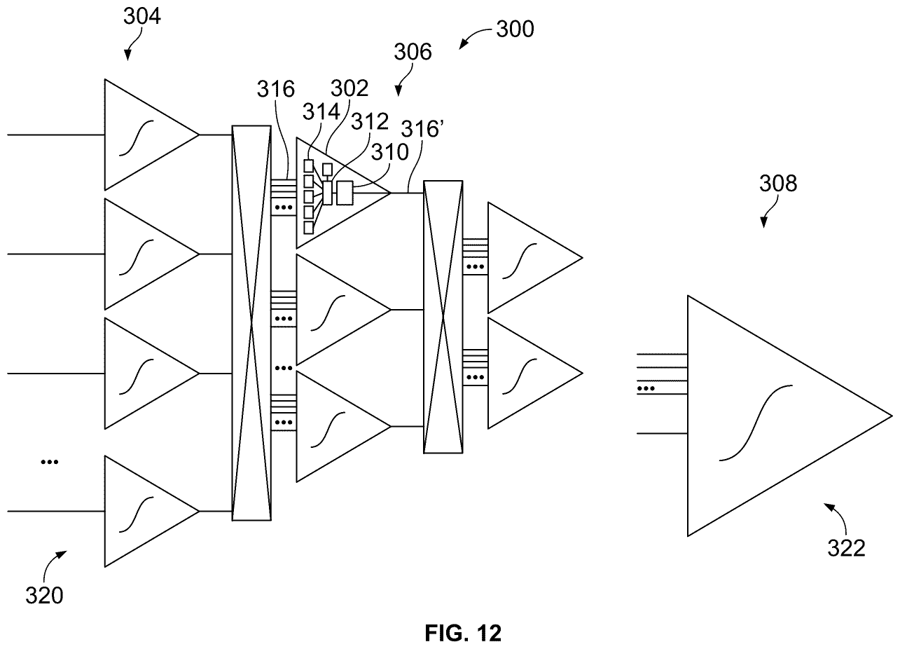

FIG. 12 is a schematic view of an artificial neural network (ANN) 300 for the detergent sensor 200 in accordance with an exemplary embodiment. The detergent sensor 200 includes one or more processors determining optical properties of the sample of the water in the fluid channel 212. For example, the NIRS 202 includes one or more processors analyzing wavelengths of the light received at the photodetector 206. In various embodiments, the NIRS 202 includes one or more processors analyzing intensity of the light received at the photodetector 206. In various embodiments, the NIRS 202 includes one or more processors analyzing transmission of the light received at the photodetector 206. In various embodiments, the NIRS 202 includes one or more processors analyzing absorbance of the light received at the photodetector 206. The optical properties of the sample of the water are affected by various factors such as the volume of the water in the appliance, the volume and/or type of materials in the laundry, the agitation level of the wash cycle, the amount of soil or contaminants suspended in the water, the amount or value of the detergent, the type of detergent, and the like. The chemical analysis performed by the NIRS 202 is used to determine one or more sensor outputs relating to the water level, the detergent level and/or the soil level, which is used to control operation of the appliance 100.

The ANN 300 can includes a series of layers, each comprising one or more artificial neurons 302 arranged in one or more neuron arrays or arrangements. A different number of neurons 302 may be in one or more of the layers and/or there may be a different number of layers in other embodiments.

The ANN 300 includes an input layer 304, one or more hidden layers 306, and an output layer 308. The input layer 304 receives external data, such as from the sensors, with each neuron representing a feature of the input data. The number of neurons in the input layer equals the number of input features. The hidden layer(s) 306 analyzes the output from the previous layer and passes it on to the next layer. There can be one or more hidden layers 306 and each neuron in the hidden layer 306 may be connected to all neurons in the adjacent layers. The hidden layers 306 use non-linear activation functions to learn to extract relevant features from the input data. The output layer 308 produces the final result of the ANNs 300 data processing. The number of neurons in the output layer 308 depends on the problem being solved, such as to determine one or more sensor outputs relating to the water level, the detergent level and/or the soil level, which is used to control operation of the appliance 100. The connections between nodes in the ANN 300 are represented by numbers called weights. Larger weights contribute more significantly to the output than other inputs.

Each neuron 302 can include or represent a register 310, a microprocessor 312, and at least one input 314. The neurons 302 can generate outputs based on one or more activation functions. The neurons 302 can receive input from another neuron 302 (e.g., the output from one neuron 302 can be the input for another neuron 302). This neuron 302 also can include a set of weights and bias as well as a summing node and Sigmoid or other non-linear activation function to process the input. The neurons 302 can be connected with each other via synaptic circuits 316, 316′. The synaptic circuits 316, 316′ can include or represent memories for storing synaptic weights.

One or more neurons 302 in the input layer 304 of the ANN 300 can receive an input 320 into the ANN 300. These neurons 302 can receive this input via the input(s) 314 of those neurons 302 in the input layer 304. The neurons 302 receive the input, apply one or more mathematical equations or relationships stored in the registers 310 (and that include the weights) to generate an output. The processors 312 of the neurons 302 apply the equations/relationships and can pass the output to another neuron 302 in the same layer 304 or in a different layer 306, 308. The output from one neuron 302 is passed along a synaptic circuit 316 to another neuron 302 and is used as input to this other neuron 302. This process continues until one or more neurons 302 in the output layer 308 generate an output 322 from the ANN 300. The synaptic circuits 316, 316′, weights stored in the synaptic circuits 316, 316′, and/or the mathematical relationships between the neurons 302 can define the model that is used to predict the runway configurations (e.g., the data).

During training of the ANN 300, labeled data may be provided as input 320 to the ANN 300. This labeled data can be encoded. The labeled data can include prior detergent type, prior detergent levels, prior agitation levels, prior contamination levels, prior chemical content, or other content related to the washing cycle. The neurons 302 process the input data as described above to generate the training output of the ANN 300. This training output can be the predicted detergent strength, the predicted agitation level, the predicted contamination level, the predicted chemical content, and the like. This prediction can then be compared the actual washing machine configurations. The past washing machine configuration predictions and the past actual washing machine configurations can be compared with each other to identify differences.

Feedback can be provided to the ANN 300 in the form of a calculated error or other indication of the differences between the past washing machine configuration predictions and the past actual washing machine configurations. Based on this error, the neurons 302 can change one or more of the synaptic circuits 316 that connect the neurons 302, the weights applied by one or more of the neurons 302, and/or the mathematical relationships between the neurons 302. For example, some synaptic circuits 316 can be changed to modified synaptic circuits 316′ such that the same input 320 would result in different neurons 302 receiving input and passing output to other neurons and generating a different output 322 from the ANN 300.

After training the ANN 300, the ANN 300 can use the trained model to predict washing machine configurations. During post-training iterations of operation of the ANN 300, additional feedback can be provided to the ANN 300 based on differences between the predicted washing machine configurations and the actual washing machine configurations. For example, after training, the ANN 300 can receive the predicted detergent strength, the predicted agitation level, the predicted contamination level, the predicted chemical content, etc., and predict the washing machine configurations. The actual detergent strength, the actual agitation level, the actual contamination level, the actual chemical content can be compared to the predicted configurations and differences (e.g., errors) can be identified. These differences can again be input into the ANN 300 to continue to change the synaptic circuits 316, 316′, neurons 302, mathematical relationships, etc. to further refine and improve the model 320 for use in continuing to improve the washing machine operations. For example, the ANN 300 may be trained and re-trained using backpropagation, which can involve adjusting model parameters (e.g., synaptic circuits 316 and/or weights) using calculated derivatives to minimize the loss function (e.g., the error). The backpropagation can be a mathematical calculation for supervised learning of the ANN 300 using gradient descent. Backpropagation can be used to calculate the gradient of the error function with respect to the weights of the ANN 300.

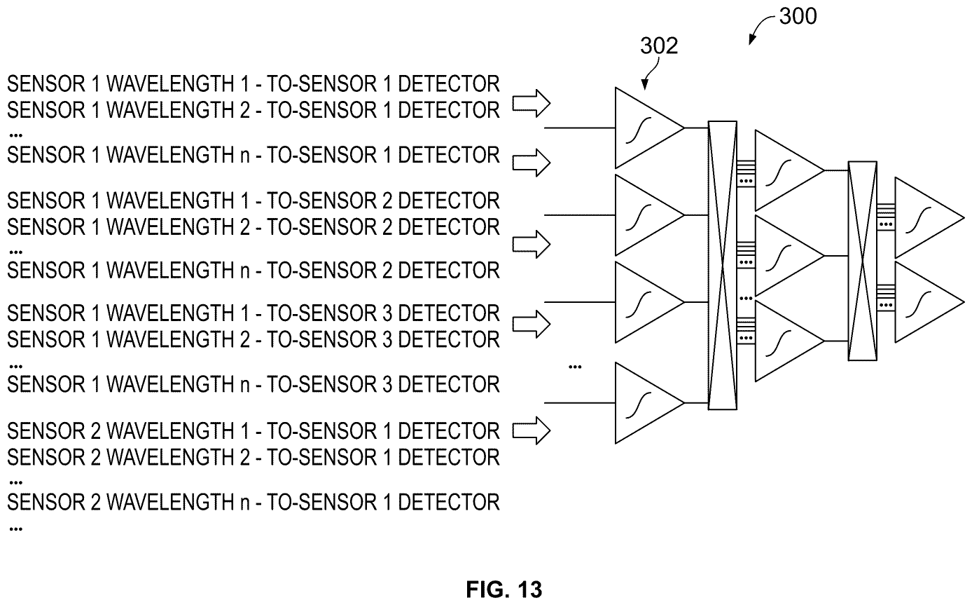

FIG. 13 is a schematic view showing exemplary inputs to the nodes 302 of the ANN 300 for the detergent sensor 200 in accordance with an exemplary embodiment. In an exemplary embodiment, the inputs to the nodes 302 include the sensor wavelengths from the various emitters 204 (LEDs) transmitted to the various photodetectors 206. The optical properties of the sample of the water are affected by various factors such as the volume of the water in the appliance, the volume and/or type of materials in the laundry, the agitation level of the wash cycle, the amount of soil or contaminants suspended in the water, the amount or value of the detergent, the type of detergent, and the like. The optical properties affect the light detected at each of the photodetectors 206. The analysis performed by the ANN 300 determines one or more sensor outputs relating to the water level, the detergent level and/or the soil level, which is used to control operation of the appliance 100.

The NIRS 202 is used during the wash cycle to control the operation of the appliance in real time. The NIRS 202 is used to enhance the appliance 100 performance and energy and water savings. The chemical analysis performed by the ANN 300 provides useful information about the contents in the water, such as the detergent and/or the soil in the water. The NIRS 202 takes spectral measurements of the sample solution and performs a chemical analysis of the sample solution using the ANN 300. For example, the ANN 300 may analyze the residual detergent content in the rinse water to allow the washing machine to run the required number of rinse cycles needed, and no more, resulting in improved performance, such as from water savings, energy savings, time savings, and the like. The NIRS 202 allows the washing machine to run more efficiently and autonomously for improved performance and enhanced consumer satisfaction.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

What is claimed is:

1. A control assembly for an appliance comprising:
a control panel for the appliance, the control panel including inputs for controlling the appliance;
a control circuit board coupled to the control panel, the circuit board configured to be operably coupled to working components of the appliance to control operation of the appliance based on the inputs from the control panel; and
a detergent sensor configured to sense wash characteristics of water in the appliance, the detergent sensor includes a near infrared spectroscope (NIRS) for determining optical properties of a sample of the water, the detergent sensor performing a chemical analysis to determine the contents of the sample based on the optical properties;
wherein the control circuit board is configured to control the working components of the appliance based on the chemical analysis performed by the detergent sensor.

2. The control assembly of claim 1, wherein the NIRS measures detergent content in the sample.

3. The control assembly of claim 1, wherein the control circuit board is configured to determine if an additional rinse cycle for the appliance is needed based on the chemical analysis.

4. The control assembly of claim 1, wherein the detergent sensor includes an artificial neural network performing the chemical analysis.

5. The control assembly of claim 4, wherein the artificial neural network analyzes at least three wavelengths of light from the NIRS to perform the chemical analysis.

6. The control assembly of claim 1, wherein the NIRS includes a light source and a photodetector receiving light from the light source.

7. The control assembly of claim 6, wherein the photodetector is operable in a reflection mode receiving light from the light source reflected by the sample.

8. The control assembly of claim 6, wherein the photodetector is operated in a transmission mode receiving light from the light source transmitted through the sample.

9. The control assembly of claim 1, wherein the detergent sensor performs the chemical analysis to determine at least one of an amount of detergent in the water and a type of detergent in the water.

10. The control assembly of claim 1, wherein the detergent sensor performs the chemical analysis to determine an amount of soil level in the water.

11. The control assembly of claim 1, wherein the detergent sensor performs the chemical analysis to determine an agitation level of the water.

12. The control assembly of claim 1, wherein the NIRS determines at least one of a transmittance, a reflectance, and an absorbance of the sample.

13. An appliance comprising:
a housing;
a drum rotatable in the housing;
a motor operably coupled to the drum to rotate the drum;
a water valve for filling the drum through a water inlet;
a water pump for draining the drum through a drain pipe; and
a control assembly for controlling the motor, the water valve, and the water pump, the control assembly including a control panel mounted to the housing, the control panel including inputs for controlling the appliance, the control assembly including a control circuit board coupled to the control panel, the control circuit board configured to be operably coupled to the motor, the water valve, and the water pump to control operation of the appliance based on the inputs from the control panel, the control assembly including a detergent sensor configured to sense wash characteristics of water in the appliance, the detergent sensor includes a near infrared spectroscope (NIRS) for determining optical properties of a sample of the water, the detergent sensor performing a chemical analysis to determine the contents of the sample based on the optical properties, wherein the control circuit board is configured to control the working components of the appliance based on the chemical analysis performed by the detergent sensor.

14. A method of controlling an appliance comprising:
providing a detergent sensor in fluid communication with an internal chamber of the appliance to sense wash characteristics of water in the appliance, wherein the detergent sensor includes a near infrared spectroscope (NIRS) for determining optical properties of a sample of the water;
performing a chemical analysis of the sample based on the optical properties to determine the contents of the sample;
sending a sensor signal from the detergent sensor to a control circuit board relating to the chemical analysis;
sending a control signal from the control circuit board to at least one working component of the appliance to control operation of the appliance based on the chemical analysis performed by the detergent sensor.

15. The method of claim 14, wherein said performing a chemical analysis includes measuring detergent content in the sample.

16. The method of claim 14, wherein said sending a control signal from the control circuit board to the at least one working component includes sending a control signal to perform an additional rinse cycle based on the chemical analysis.

17. The method of claim 14, wherein said performing a chemical analysis includes performing the chemical analysis using an artificial neural network to analyze the optical properties.

18. The method of claim 14, wherein said providing a detergent sensor includes providing a light source and a photodetector for the NIRS, the photodetector receiving light from the light source directed at the sample, wherein the photodetector is operable in at least one of a reflection mode receiving light from the light source reflected by the sample and a transmission mode receiving light from the light source transmitted through the sample.

19. The method of claim 14, wherein said performing a chemical analysis includes determining at least one of an amount of detergent in the water and a type of detergent in the water.

20. The method of claim 14, wherein said performing a chemical analysis includes determining at least one of a transmittance, a reflectance, and an absorbance of the sample.
